(19) United States
(12) Patent Application Publication
(10) Pub. No.: US 2025/0332258 A1
(43) Pub. Date: Oct. 30, 2025

(54) SWITCHABLE CAR-T THERAPIES FOR TREATING HUMAN CANCERS

(71) Applicant: THE SCRIPPS RESEARCH INSTITUTE, La Jolla, CA (US)

(72) Inventors: Travis Young, La Jolla, CA (US); Eduardo Laborda, San Diego, CA (US)

(21) Appl. No.: 18/715,941

(22) PCT Filed: Dec. 6, 2022
(86) PCT No.: PCT/US22/81010

§ 371 Date: Jun. 3, 2024

Related U.S. Application Data
(60) Provisional application No. 63/286,868, filed on Dec. 7, 2021.

(51) Int. Cl. A61K 40/42; A61K 40/11; A61K 40/31; C07K 14/725; C07K 16/18; C07K 16/28

(52) U.S. Cl. CPC A61K 40/4242; A61K 40/11; A61K 40/31; C07K 14/7051; C07K 16/18; C07K 16/2803; A61K 2239/23; C07K 2317/24; C07K 2317/55; C07K 2317/622; C07K 2319/00

(57) ABSTRACT

The invention provides methods of treating CD-19 positive malignancies in human subjects with suitable doses of switchable CAR-T cells and complementary switch molecules.

### CROSS-REFERENCE TO RELATED APPLICATIONS

The subject patent application is a § 371 U.S. national phase filing of PCT International Patent Application No. PCT/US2022/081010 (filed Dec. 6, 2022; now expired), which claims the benefit of priority to U.S. Provisional Patent Application No. 63/286,868 (filed Dec. 7, 2021; now expired). The full disclosure of the priority applications are incorporated herein by reference in their entirety and for all purposes.

### SEQUENCE LISTING

The instant application contains a Sequence Listing, which is submitted in .xml format and is hereby incorporated by reference in its entirety. Said .xml file is named “2120_1PC_SeqListing”, was created on Feb. 22, 2023, and is 17 KB bytes in size.

### BACKGROUND OF THE INVENTION

Despite the tremendous clinical benefits, adverse events associated with CAR-T cell therapy remain a challenge. Most frequent adverse events are cytokine release and immune effector cell-associated neurotoxicity syndromes due to the inability to modulate the level of activity of current CAR-T cell products after administration to patients. Additional challenges include on target, off tumor toxicities and antigen loss mediated relapse of disease.

To address these challenges, a “switchable” CAR-T (sCAR-T) platform has been developed, where the activity of the sCAR-T cells is controlled by an antibody-based switch. The switch targets the tumor antigen, and the sCAR recognizes a unique peptide engrafted on the switch. The switch creates a bridge between the sCAR-T cell and the tumor cell, activating the sCAR-T cells and inducing tumor cell killing. Combined, the switch and sCAR-T cells afford complete elimination of tumors in xenograft and syngeneic models, but individually, each is designed to be inactive. A short half-life of the switch allows for a rapid modulation of sCART-cell activity through the switch dosing. Moreover, by swapping different switches, sCAR-T cells can be modularly redirected against other tumor targets. It was shown the cyclical on/off stimulation of the sCAR-T cells affords improved memory and persistence of the sCAR-T cells.

There is a strong need in the art for actual application of the sCAR-T platform in treating human subjects. The present invention is directed to addressing this and other unmet needs.

### SUMMARY OF THE INVENTION

In one aspect, the invention provides methods of treating, arresting growth of, and/or promoting regression of, a CD19-positive malignancy in a human subject. The methods involve administering to the subject (a) a chimeric antigen receptor-T cell switch molecule (CAR-T switch) comprising an anti-CD19 Fab antibody that comprises a light chain variable region and a heavy chain variable region sequence set forth as SEQ ID NOs:2 and 3, respectively, and (b) a complementary CAR-T cell comprising a CAR sequence set forth in SEQ ID NO:6; thereby treating, arresting growth of, and/or promoting regression of, the B cell malignancy in the subject. In some embodiments, the employed anti-CD19 Fab antibody contains a light chain sequence and a heavy chain sequence set forth as SEQ ID NOs:15 and 16, respectively. In some embodiments, the subject is administered with one dose of the CAR-T cell at the beginning of the treatment, and multiple doses of the CAR-T switch during the course of the treatment. In some of these embodiments, the subject is infused with one dose of the CAR-T cells, followed by one or multiple cycles of infusion of the CAR-T switch. Each of the cycles of infusion contains an “on” phase of daily infusion of the CAR-T switch for about 5 to about 9 days, and an “off” phase of no CAR-T administration for about 14 to about 28 days.

In various embodiments, the dose of the CAR-T cells administered to the subject is about 60×10⁶, 80×10⁶, 100×10⁶, 120×10⁶, 140×10⁶, 160×10⁶, 180×10⁶, 200×10⁶, 300×10⁶, 400×10⁶, 500×10⁶, 600×10⁶, 700×10⁶, 800×10⁶, 900×10⁶, 1000×10⁶ or more cells. In some embodiments, the dose of the CAR-T cells administered to the subject is from about 0.35×10⁸ to about 14×10⁸ cells. In some of these embodiments, the dose of the CAR-T cells administered to the subject is from about 1.4×10⁸ to about 7×10⁸ cells. In one embodiment, the dose of the CAR-T cells administered to the subject is about 1.4×10⁸ cells.

In some methods of the invention, a dose of the CAR-T switch administered to the subject is from about 0.01 mg to about 0.1 mg per kg of body weight. In various embodiments, a dose of the CAR-T switch administered to the subject can be about 0.01 mg, 0.025 mg, 0.03 mg, 0.04 mg, 0.045 mg, 0.05 mg, 0.055 mg, 0.06 mg, 0.065 mg, 0.070 mg, 0.075 mg, 0.085 mg, or 0.095 mg per kg of body weight. In some embodiments, a dose of the CAR-T switch administered to the subject is from about 0.045 mg to about 0.075 mg per kg of body weight. In one embodiment, a dose of the CAR-T switch administered to the subject is about 0.06 mg per kg of body weight.

In some methods of the invention, the CD19-positive malignancy in the subject to be treated is a CD19-positive B cell cancer. In some methods, the CD19-positive malignancy to be treated is a relapsed/refractory B cell malignancy.

In a related aspect, the invention provides methods for treating, arresting growth of, and/or promoting regression of, a CD19-positive relapsed/refractory B cell malignancy in a human subject. These methods entail (a) administering to the subject one dose of about 0.35×10⁸ to about 7×10⁸ CAR-T cells that contain a CAR sequence set forth in SEQ ID NO:6, and then (b) administering to the subject a CAR-T switch molecule, which is a modified anti-CD19 Fab antibody containing a light chain sequence and a heavy chain sequence respectively set forth as SEQ ID NOs:15 and 16, during one or more infusion cycles that each contains (i) an “on” phase of about 5 to about 9 days and (ii) an “off” phase of about 14 to about 28 days, with a daily dose of about 0.045 mg to about 0.075 mg per kg of body weight of the CAR-T infused to the subject during the “on” phase. Preferably, the administered CAR-T cells are autologous to the subject. In some embodiments, the infused dose of the CAR-T cells is about 1.4×10⁸ cells, and the daily dose of the CAR-T switch infused during the “on” phase is about 0.06 mg per kg of body weight. In some of these embodiments, the “on” phase is about 7 days, and the “off” phase is about 21 days. In various embodiments, the number of cycles of CAR-T switch infusion can be 2, 3, 4, 5, 6, 7, 8, 9, 10 or more.

In some of the methods, the subjects are to be treated with lymphodepletion preconditioning prior to being administered the CAR-T cells and the switch molecule. In some of these embodiments, lymphodepletion preconditioning in the subjects can be achieved via chemotherapy with cyclophosphamide and fludarabine. In various embodiments, the CD19-positive relapsed/refractory B cell malignancy to be treated in the subject can be, e.g., diffuse large B-cell lymphoma (DLBCL), follicular lymphoma (FL), chronic lymphocytic leukemia (CLL), small lymphocytic lymphoma (SLL), mantle cell lymphoma (MCL), marginal zone lymphoma (MZL), hairy cell leukemia (HCL), primary intraocular lymphoma, Burkitt lymphoma, or Waldenström macroglobulinemia.

A further understanding of the nature and advantages of the present invention may be realized by reference to the remaining portions of the specification and claims.

### DESCRIPTION OF THE DRAWINGS

FIG. 1 shows in vivo studies with MABEL based approach to determine first-in-human (FIH) dose.

FIG. 2 shows simulated human equivalent doses of CAR-T switch molecule SWI019.

FIG. 3 depicts CT scan that shows treatment effect in a patients

FIG. 4 shows resolution of toxicity in treated patients.

FIG. 5 is a schematic illustration of a dose escalation Phase I trial of a sCAR-T therapy. A. Design of phase I dose escalation study. B. Overview of treatment schedule.

FIG. 6 shows a BAYDE model dose recommendations by clinical utility index.

### DETAILED DESCRIPTION

The invention is predicated in part on the studies undertaken by the present inventors, detailed below, to determine appropriate doses of a switchable CAR-T therapy in human patients. In particular, through a dose escalation Phase I human clinal trial, the inventors were able to determine suitable doses of the switch and the corresponding CAR-T cell of a specific CD19 targeting sCAR-T platform in the treatment of several CD19-positive malignancies.

The present invention accordingly provides methods and dosage regimens for treating human patients afflicted with various cancers, e.g., CD19-expressing tumors, with the sCAR-T platforms described herein. Unless otherwise stated, the present invention can be performed using standard procedures, as described, for example in Methods in Enzymology, Volume 289: Solid-Phase Peptide Synthesis, J. N. Abelson, M. I. Simon, G. B. Fields (Editors), Academic Press; 1st edition (1997) (ISBN-13:978-0121821906); U.S. Pat. Nos. 4,965,343, and 5,849,954; Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2nd ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998).

The following sections provide additional guidance for practicing the compositions and methods of the present invention.

### I. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

It is noted that, as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements or use of a “negative” limitation.

The term “antibody” typically refers to polypeptide chain(s) which exhibit a strong monovalent, bivalent or polyvalent binding to a given antigen, epitope or epitopes. Unless otherwise noted, antibodies or antibody fragments used in the invention also encompass certain immunoglobulin molecules or variant antibodies that do not possess specific antigen-binding activities, e.g., catalytic antibodies. immunoglobulin s can have sequences derived from any vertebrate species. They can be generated using any suitable technology, e.g., hybridoma technology, ribosome display, phage display, gene shuffling libraries, semi-synthetic or fully synthetic libraries or combinations thereof. Unless otherwise noted, the term “antibody” as used in the present invention includes intact antibodies, antibody fragments and other designer antibodies that are described below or well known in the art (see, e.g., Serafini, J Nucl. Med. 34:533-6, 1993).

An intact “antibody” typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Each heavy chain of an antibody is comprised of a heavy chain variable region (V_{H}) and a heavy chain constant region. The heavy chain constant region of most IgG isotypes (subclasses) is comprised of three domains, C_{H1}, C_{H2} and C_{H3}, some IgG isotypes, like IgM or IgE comprise a fourth constant region domain, C_{H4} Each light chain is comprised of a light chain variable region (V_{L}) and a light chain constant region. The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system and the first component (Clq) of the classical complement system.

The V_{H} and V_{L} regions of an antibody can be further subdivided into regions of hypervariability, also termed complementarity determining regions (CDRs), which are interspersed with the more conserved framework regions (FRs). Each V_{H} and V_{L} is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined by, e.g., Kabat et al., Sequences of Proteins of Immunological Interest, U.S. Department of Health and Human Services, U.S. Government Printing Office (1987 and 1991).

As used herein, an antibody fragment (or antigen binding fragment of an antibody) refers to any proteins or polypeptides that contain at least one antibody-derived V_{H}, V_{L}, or C_{H} immunoglobulin domain in the context of other non-immunoglobulin, or non-antibody derived components. Such molecules include, but are not limited to (i) F_{c}-fusion proteins of binding proteins, including receptors or receptor components with all or parts of the immunoglobulin C_{H} domains, (ii) binding proteins, in which V_{H} and or V_{L} domains are coupled to alternative molecular scaffolds, or (iii) molecules, in which immunoglobulin V_{H}, and/or V_{L}, and/or C_{H} domains are combined and/or assembled in a fashion not normally found in naturally occurring antibodies or antibody fragments.

“Humanized” forms of non-human (e.g., rodent, e.g., murine or rabbit) immunoglobulins are immunoglobulins which contain minimal sequences derived from non-human immunoglobulin. For the most part, humanized immunoglobulins are human immunoglobulins (recipient antibody) in which residues from a hypervariable region of the recipient are replaced by residues from a hypervariable region of a non-human species (donor antibody) such as mouse, rat, hamster, rabbit, chicken, bovine or non-human primate having the desired specificity, affinity, and capacity. In some instances, Fv framework region (FR) residues of the human immunoglobulin are also replaced by corresponding non-human residues. Furthermore, humanized antibodies can comprise residues which are not found in the recipient antibody or in the donor antibody. These modifications are made to further refine antibody performance. In general, the humanized immunoglobulin will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized immunoglobulin optionally also will comprise at least a portion of an immunoglobulin constant region (Fc), typically that of a human immunoglobulin. For further details, see Jones et al. (1986) Nature 321:522-525; Riechmann et al. (1988) Nature 332:323-329; and Presta (1992) Curr. Op. Struct. Biol. 2:593-596.

The term “human immunoglobulin”, as used herein, is intended to include immunoglobulins having variable and constant regions derived from human germline immunoglobulin sequences. The human immunoglobulins of the invention can include amino acid residues not encoded by human germline immunoglobulin sequences (e.g., mutations introduced by random or site-specific mutagenesis in vitro or by somatic mutation in vivo), for example in the CDRs and in particular CDR3. However, the term “human immunoglobulin”, as used herein, is not intended to include immunoglobulins in which CDR sequences derived from the germline of another mammalian species, such as a mouse, have been grafted onto human framework sequences.

Lymphodepleting (LD) chemotherapy or lymphodepleting preconditioning refers to a therapy administered to subjects before CAR-T cell infusion, in order to deplete endogenous T-cells (and Tregs), so that they are not going to antagonize/suppress and allow expansion/proliferation of the infused CAR-T cells. One example of LD preconditioning is to administer the subjects with Fludarabine+Cyclophosphamide (FluCy).

The term “specific binding” or “specifically binds to” or is “specific for” refers to the binding of a binding moiety to a binding target, such as the binding of an immunoglobulin or small molecule agent to a target molecule or antigen, e.g., an epitope on a particular polypeptide, peptide, or other target (e.g. a glycoprotein target), and means binding that is measurably different from a non-specific interaction (e.g., a non-specific interaction can be binding to bovine serum albumin or casein). Specific binding can be measured, for example, by determining binding of a binding moiety (e.g., a small molecule agent), or an immunoglobulin, to a target molecule compared to binding to a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target.

The term “specific binding” or “specifically binds to” or is “specific for” a particular target molecule or an epitope on a particular target molecule can be exhibited, for example, by a molecule having a K_{d} for the target of at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater. In certain instances, the term “specific binding” refers to binding where a binding moiety binds to a particular target molecule or epitope on the target molecule without substantially binding to any other molecule or epitope.

The term “fusion” is used herein to refer to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini. The term “fusion” is used herein to refer to the combination of amino acid sequences of different origin.

The term “epitope” includes any molecular determinant capable of specific binding to an immunoglobulin. In certain aspects, epitope determinants include chemically active surface groupings of molecules such as amino acids, sugar side chains, phosphoryl, or sulfonyl, and, in certain aspects, can have specific three dimensional structural characteristics, and/or specific charge characteristics. An epitope is a region of an antigen that is bound by an immunoglobulin. A “binding region” is a region on a binding target bound by a binding molecule.

The term “target” or “binding target” is used in the broadest sense and specifically includes polypeptides, without limitation, nucleic acids, carbohydrates, lipids, cells, and other molecules with or without biological function as they exist in nature. In some specific embodiments, the term target refers to a cell surface molecule on a target cell, e.g., a tumor cell.

The term “antigen” refers to an entity or fragment thereof, which can bind to an immunoglobulin or trigger a cellular immune response. An immunogen refers to an antigen, which can elicit an immune response in an organism, particularly an animal, more particularly a mammal including a human. The term antigen includes regions known as antigenic determinants or epitopes, as defined above.

A nucleic acid is “operably linked” when it is placed in a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

“Percent (%) amino acid sequence identity” with respect to a peptide or polypeptide sequence, i.e., an scFV antibody polypeptide sequence or a GCN4 derived peptide identified herein, is defined as the percentage of amino acid residues in a candidate sequence that are identical with the amino acid residues in the specific peptide or polypeptide sequence after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Two sequences are “substantially identical” if they have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the well-known sequence comparison algorithms or by manual alignment and visual inspection.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. For example, a subject or mammal is successfully “treated” for cancer, if, after receiving a treatment of the present invention, the subject shows observable and/or measurable reduction in or absence of one or more of the following: reduction in the number of cancer cells or absence of the cancer cells; reduction in the tumor size; inhibition (i.e., slowing to some extent and preferably stopping) of cancer cell infiltration into peripheral organs, including the spread of cancer into soft tissue and bone; inhibition (i.e., slowing to some extent and preferably stopping) of tumor metastasis; inhibition, to some extent, of tumor growth; and/or relief to some extent of one or more of the symptoms associated with the specific cancer; reduced morbidity and/or mortality, and improvement in quality of life issues.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “contacting” has its normal meaning and refers to combining two or more agents (e.g., polypeptides or phage), combining agents and cells, or combining two populations of different cells. Contacting can occur in vitro, e.g., mixing an antibody and a cell or mixing a population of antibodies with a population of cells in a test tube or growth medium. Contacting can also occur in a cell or in situ, e.g., contacting two polypeptides in a cell by co-expression in the cell of recombinant polynucleotides encoding the two polypeptides, or in a cell lysate. Contacting can also occur in vivo inside a subject, e.g., by administering an agent to a subject for delivery the agent to a target cell.

The term “subject” in general refers to both human and non-human animals (especially non-human mammals). Unless otherwise noted, the term preferably refers to human patients in connection with the disclosed therapeutic methods.

Artificial T cell receptors (also known as chimeric T cell receptors, chimeric immunoreceptors, chimeric antigen receptors (CARs) or T-bodies) are engineered receptors, which graft an arbitrary specificity onto an immune effector cell. Typically, these receptors are used to graft the specificity of a monoclonal antibody onto a T cell; with transfer of their coding sequence facilitated by retroviral or lentiviral vectors or by transposons. CAR-engineered T cells (also abbreviated CAR-T cells or CAR⁺ T cells herein) are genetically engineered T cells armed with chimeric receptors whose extracellular recognition unit is comprised of an antibody-derived recognition domain and whose intracellular region is derived from one or more lymphocyte stimulating moieties. The structure of the prototypic CAR is modular, designed to accommodate various functional domains and thereby to enable choice of specificity and controlled activation of T cells. The preferred antibody-derived recognition unit is a single chain variable fragment (scFv) that combines the specificity and binding residues of both the heavy and light chain variable regions of a monoclonal antibody. The most common lymphocyte activation moieties include a T-cell costimulatory (e.g. CD28 and/or 4-1BB) domain in tandem with a T-cell triggering (e.g. CD3zeta) moiety. By arming effector lymphocytes (such as T cells and natural killer cells) with such chimeric receptors, the engineered cell is re-directed with a pre-defined specificity to any desired target antigen, in a non-HLA restricted manner. CAR constructs are introduced ex vivo into T cells from peripheral lymphocytes of a given patient using retroviral or lentiviral vectors or transposons. Following infusion of the resulting CAR-engineered T cells back into the patient, they traffic, reach their target site, and upon interaction with their target cell or tissue, they undergo activation and perform their predefined effector function. Therapeutic targets for the CAR approach include cancer and HIV-infected cells, or autoimmune effector cells.

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

As used herein, a sCAR-T platform refers to a CAR-T switch molecule and a complementary CAR-T cell (aka sCAR-T cell as used herein). The CAR-T switch molecule (“CAR-T switch”) contains a targeting moiety (e.g., an antibody or antigen-binding fragment thereof) that is capable of specifically binding to a target molecule on the surface of a target cell (e.g., a tumor cell). The CAR-T switch is also able to bind to the CAR of the complementary CAR-T cell. Typically, the extracellular domain of the CAR of the CAR-T cell contains an antibody moiety (e.g., a scFv) that specifically recognizes a CAR-ID domain (e.g., a peptide or a small molecule) in the CAR-T switch.

### II. Switchable CAR-T Platforms for Treating Human Patients

The present invention provides sCAR-T therapies for treating human patients afflicted with various cancers, tumors or malignancies. In some embodiments, the methods are intended to treat or ameliorate the symptoms of a CD-19 positive malignancy in the patients. In related embodiments, the novel therapies of the invention are directed to promoting tumor regression and/or to arresting tumor growth in the subjects. In some embodiments, the subjects to be treated with the therapies of the invention are preconditioned with lymphodepleting chemotherapy (aka “LD preconditioning”). This LD preconditioning is an essential step before the subjects can be actually infused with the CAR-T cells. It creates a “favorable” environment for CAR T-cell expansion and survival in vivo, presumably by eliminating regulatory T cells. LD preconditioning can lead to the upregulation of tumor immunogenicity and improve disease control. It has been shown that LD preconditioning works to promote homeostatic proliferation of adoptively transferred T cells via increases in the pro-survival/proliferation cytokines, interleukin (IL)-7 and IL-15, and in conjunction with a lack of competition with wildtype T cells. Pre-treatment lymphodepletion preconditioning can be readily performed with methods well known in the art, e.g., via cyclophosphamide and fludarabine conditioning chemotherapy as exemplified herein. See, e.g., Paplham et al., Leuk Res Rep. 3: 28-31, 2014; Bot et al., Blood 126: 4426, 2015; Hirayama et al., Blood 133: 1876-1887, 2019; Hay and Turtle, Drugs 77: 237-245, 2017; and Yakoub-Agha et al., Haematologica. 105: 297-316, 2020.

In general, subjects to be treated (optionally after undergoing LD chemotherapy) are administered a switchable CAR-T cell platform (sCAR-T) that is designed for the treatment of a specific cancer that the subject is afflicted with, e.g., a CD19-positive B cell malignancy. The switchable CAR-T cell platform contains (a) a CAR-T switch (also referred to herein as CAR-T switch molecule, including CAR-T switch polypeptide or CAR-T switch compound) that can bind to the CAR of a CAR-T cell and also specifically targets a cell surface molecule on a tumor cell, and (b) a complementary CAR-T cell that contains a CAR that can be bound by the switch. When treating human subjects, the administered CAR-T switch and the complementary CAR-T cells are preferably human or humanized. For example, the CAR can be a humanized polypeptide, and the T cell in which the CAR is to be expressed can be human cells. In some of these embodiments, the T cells expressing the humanized CAR are autologous T cells isolated from the specific human subject to be treated. In general, the administration can be performed in accordance with standard protocols of immunotherapy or the more detailed guidance provided herein. In some preferred embodiments, the CAR-T switch and the complementary CAR-T cells are administered to the subject by infusion. In some embodiments, the sCAR-T treatment methods described herein can be used in combination with other known therapies or therapeutic agents for treating cancers, e.g., chemotherapy, hormone therapy, radiation therapy, or surgery.

In some methods, the subject to be treated can be administered with more than one CAR-T switches, along with the complementary CAR-T cells, that target different surface molecules on a tumor cell. In some preferred embodiments, the different CAR-T switch molecules contain the same CAR-ID domain, which allows the different switches to interact with the same complementary CAR-T cell. Such a treatment is especially beneficial for heterogeneous tumors. For example, subjects afflicted with leukemias and lymphomas can be treated with a pharmaceutical composition that contains (a) both a CD19-targeting CATR-T switch plus a CD20- or CD22-targeting switch, and (b) the complementary CAR-T cells. In these embodiments, the CAR-T switches can be administered sequentially or simultaneously. A second switch targeting a second cell surface molecule on the target cell may be administered after down regulation of a first cell surface molecule on the target cell that is targeted by a first switch.

In a related aspect, the methods of the invention can be used in general for engrafting or expanding CAR-T cells in a subject. Typically, the subject is one afflicted with a disease or condition (e.g., a cancer) that the CAR-T cells are intended to treat. In these methods, the subject is administered with (a) a CAR-T switch that contains (i) a chimeric antigen receptor-interacting domain (CAR-ID) and (ii) a targeting moiety that is specific for a molecule manifesting the disease or condition afflicted by the subject (e.g., a tumor cell surface molecule), and (b) a complementary CAR-T cell that has in the extracellular domain of its CAR a single-chain variable fragment (scFv) that specifically binds to the CAR-ID.

Typically, the switchable CAR-T cell platform to be used in the methods of the invention contains one or more CAR-T switch molecules and one or more complementary CAR-T cells. In some embodiments, the administered one or more switches are complementary to the same CAR-T cell. Typically, the CAR-T switch to be administered to the subject contains a chimeric antigen receptor interacting domain (CAR-ID) and a targeting domain or targeting moiety. The CAR-ID specifically binds to the extracellular domain of the CAR on the complementary CAR-T cell. The CAR-ID of the CAR-T switches can be any substance that may be fused, conjugated, or otherwise attached to a targeting moiety described herein (e.g., an anti-CD19 antibody or antigen binding portion thereof), such that the CAR-ID is capable of being bound by the CAR of the CAR-T cells. For example, the CAR-ID may be a CAR-binding protein, a CAR-binding peptide, a CAR-binding small molecule. In some preferred embodiments, the CAR-ID contains a yeast transcription factor GCN4 peptide or a derivative or a homolog thereof. See, e.g., Hinnebusch and Fink, Proc Natl Acad Sci USA 80:5374-8, 1983; Arndt et al., Proc Natl Acad Sci USA 83: 8516-20, 1986; WO2015057834 and WO2015057852. In some of these embodiments, the yeast transcription factor GCN4 peptide contains a GCN4(7P14P) peptide sequence or epitope as described in Berger et al. FEBS Letters 450: 149-153, 199; and Zahnd, C., et al., J. Biol. Chem. 279: 18870-18877, 2004. As exemplification, the GCN4 derived peptide in the CAR-ID can contain the sequence NYHLENEVARLKKL (SEQ ID NO:1) or RMKQLEPKVEELLPKNYHLENEVARLKKLVGER (SEQ ID NO:13).

The targeting moiety of the employed CAR-T switch can bind to any target molecule that is present on the surface of a target tumor cell, e.g., CD19 as exemplified herein. Preferably, the target molecule can contain an antigen. In various embodiments, the target molecule can be a protein, a lipid moiety, a glycoprotein, a glycolipid, a carbohydrate, a polysaccharide, a nucleic acid, an MHC-bound peptide, or a combination thereof. In some preferred embodiments, the targeting moiety is a targeting polypeptide such as a targeting antibody or antigen-binding fragment thereof (e.g., an Fab as exemplified herein). The targeting antibody can be human, fully human, humanized, human engineered, non-human, and/or chimeric antibody. In some embodiments, a non-human antibody to be used in the CAR-T switch can be humanized to reduce immunogenicity to humans, while retaining the specificity and affinity of the parental non-human antibody. When treating human patients, the targeting antibody is preferably a humanized or human antibody. In various embodiments, the targeting antibody can specifically bind to different target molecules on tumor cells, e.g., CD19, Her2, CLL1, CD33, CD123, EGFR, EGFRvIII, CD20, CD22, CS1, BCMA, CEA or a fragment thereof.

In some preferred embodiments, the targeting moiety of the CAR-T switch is an antibody or antigen-binding fragment that contains humanized heavy and/or light chain sequences. Some specific examples of tumor-targeting CAR-T switches that can be used and/or humanized for use in the methods of the invention are described, e.g., in PCT/US2017/057460, PCT/US2014/060713, PCT/US2014/060684, PCT/US2016/024524, PCT/US2016/027997, and PCT/US2016/027990.

In some embodiments, the CAR-ID and the targeting moiety are fused together. In some of these embodiments, a structural component (e.g., a peptide terminus) of the CAR-ID is joined with or liked to a terminus of a polypeptide targeting moiety (e.g., a humanized anti-CD19 antibody or an antigen binding fragment thereof). In some embodiments, the CAR-ID and the targeting moiety are fused together via a linker. In some embodiments, the CAR-ID is attached to the targeting moiety in a site-specific manner. Attachment in a site-specific manner may entail attaching the CAR-ID to a predetermined site on the targeting moiety, e.g., light chain N-terminus of a targeting antibody as in the switch molecule exemplified herein. In some embodiments, site-specific attachment can entail attaching the CAR-ID to an unnatural amino acid in the targeting moiety. As a specific exemplification, the CAR-T switch employed in the methods of the invention is a CD19 targeting molecule, SWI019. It is a modified Fab antibody that contains a GCN4 derived peptide NYHLENEVARLKKL (SEQ ID NO:1) fused via a GGGGS (SEQ ID NO:14) linker to the N terminus of the light chain of a humanized anti-CD19 Fab molecule. The resulting GCN peptide fused light chain variable region sequence of the Fab molecule is shown SEQ ID NO:2. Along with this light chain sequence, the SWI019 switch (modified Fab molecule) also has a heavy chain variable region sequences shown in SEQ ID NO:3. Including the constant regions, the light chain and heavy chain sequences of SWI019 are shown in SEQ ID NOs:15 and 16, respectively. More detailed information of the structure of this CAR-T switch is described in, e.g., U.S. Pat. No. 11,174,306; and Rodgers DT, et al., PNAS, 2016; 113(4):E459-68.

Along with the CAR-T switch, a complementary CAR-T cell is also administered to a subject for the treatment of a cancer. Typically, the CAR-T cells contain a chimeric antigen receptor (CAR) that contains an extracellular domain, a transmembrane domain and an intracellular signaling domain. The extracellular domain is capable of specifically binding to the CAR-ID (e.g., a GCN4, Flag, K4, or E4 peptide, or a small molecule such as FITC) of the employed CAR-T switch. In some preferred embodiments, the extracellular domain of the CAR contains an antibody or antibody fragment (e.g., a scFv) that binds to the CAR-ID of the switch. The antibody may be human, fully human, humanized, human engineered, non-human, and/or chimeric antibody. For treating human patients, the antibody in the CAR of the CAR-T cell is preferably human or humanized. A number of known protein transmembrane domains can be used in the CAR of the CAR-T cells. In some embodiments, the transmembrane domain can be the transmembrane domain of CD8 or CD28. In general, the intracellular signaling domain can contain signaling domains such as CD3ζ, FcR-γ, and Syk-PT as well as co-signaling domains such as CD28, 4-1BB, and CD134. In some embodiments, the intracellular signaling domain of the CAR can contain (a) a CD3-zeta domain, plus (b) a CD28 domain, a 4-1BB domain, or both a CD28 domain and a 4-1BB domain. In some embodiments, a hinge region is present in the CAR to connect the extracellular domain with the transmembrane domain.

Depending on the CAR-ID in the employed CAR-T switch, various CAR-ID binding moieties can be present in the extracellular domains of the complementary CAR-T cells. In some embodiments, the extracellular domain of the CAR can contain an antibody or antibody fragment that recognizes a yeast transcription factor GCN4 or a fragment thereof. See, e.g., Rodgers et al., Proc Natl Acad Sci USA 113:E459-E468, 2016. In some embodiments, the extracellular domain of the CAR of the CAR-T cells can contain an anti-fluorescein isothiocyanate (FITC) antibody or a FITC-binding portion thereof. The anti-FITC antibody can be an anti-FITC scFv. In some embodiments, the extracellular domain of the CAR of the CAR-T cells can contain an antibody or fragment that recognizes a synthetic (non-naturally-occurring) peptide. For example, the antibody in the CAR can be one that specifically recognizes a FLAG® tag or a fragment thereof. In some embodiments, the extracellular domain of the CAR can contain an anti-HTP antibody or a fragment thereof.

When treating a human subject, the CAR of the administered CAR-T cells should preferably be human or humanized to reduce immunogenicity to humans. In some embodiments, the extracellular domain of the CAR of the administered CAR-T cells contain a humanized scFv that binds to the CAR-ID of the co-administered switch. The humanized scFv can comprise a humanized VH (variable heavy chain) sequence with non-human (e.g., murine) CDRs grafted onto a human immunoglobulin framework. In some embodiments, the extracellular domain of the CAR can contain a humanized anti-GCN4 scFv. Such scFv molecules can be derived from the anti-GCN4 scFv clone 52SR4 described in Zahnd et al., J. Biol. Chem. 279:18870-77, 2004. The humanized anti-GCN4 scFv (e.g., a humanized version of clone 52SR4) can contain a humanized light chain, a humanized heavy chain, or a humanized light chain and a humanized heavy chain. The humanized anti-GCN4 (e.g., a humanized version of clone 52SR4) can contain a humanized VH (variable heavy chain) sequence with non-human (e.g., murine) CDRs transplanted onto a human immunoglobulin framework. A specific example of such humanized anti-GCN4 scFv molecules that can be used in the CAR-T cells of the invention contains the sequence set forth in SEQ ID NO:7, as in the sCAR-T cell exemplified herein.

The complementary and inert CAR-T cells to be used in the invention can be prepared in accordance with methods well known in the art or specific protocols exemplified in, e.g., WO2018/075807, WO2015057834, WO2015057852, and Marcu-Malina et al., Expert Opinion on Biological Therapy, Vol. 9, No. 5. In general, recombinant technology can be used to introduce CAR-encoding genetic material into any suitable T-cells, e.g., human T cells such as central memory T-cells. As exemplification, the CAR-T cells to be used in the methods of the invention can be generated by transduction of human T cells with a lentiviral vector expressing the engineered CAR (e.g., a humanized CAR). A number of humanized CAR sequences and CAR-T cells harboring such humanized CAR sequences are known in the art. They can all be readily employed and/or adapted for use in the methods of the invention. See, e.g., WO2018/075807; Li et al., Biomarker Research 8: 36, 2020; and Maude et al., Blood 128: 217, 2016.

As a specific exemplification, the CAR-T switch to be used in the methods of the invention can contain a humanized anti-CD19 antibody or antigen-binding fragment thereof (e.g., a Fab) that is fused to a GCN4 peptide. The humanized anti-CD19 antibody can contain a light chain variable region sequence and a heavy chain variable region sequence that are identical to or substantially identical to (e.g., at least 95%, 96%, 97%, 98% or 99%) to SEQ ID NOs:2 and 3, respectively. The GCN4 peptide CAR-ID can contain an amino acid sequence that is identical to or substantially identical to (e.g., at least 95%, 96%, 97%, 98% or 99%) to SEQ ID NO:1, and is fused to the anti-CD19 antibody at the N-terminus of the light chain. The complementary CAR-T cells to be employed along with this CAR-T switch contain in its extracellular domain a humanized anti-GCN4 antibody or antigen-binding fragment thereof (e.g., a scFv). The light chain variable region sequence and heavy chain variable region sequence of the anti-GCN4 antibody are shown in SEQ ID NOs:4 and 5, respectively. A specific anti-GCN4 scFv molecule that has these two variable region sequences connected by a GS linker is shown in SEQ ID NO:7. In this CAR, a hinge region (SEQ ID NO:8) is present to connect the C-terminus of the scFv to the N-terminus of the transmembrane domain (SEQ ID NO:9). The intracellular domain of the CAR contains from the N-terminus to the C-terminus a CD28 domain (SEQ ID NO:10), a 4-1BB domain (SEQ ID NO:11) and a CD3-zeta domain (SEQ ID NO:12). The amino acid sequence of the entire CAR molecule is shown in SEQ ID NO:6. Synthesis and production of this CD-19 targeting humanized CAR-T switch and the complementary CAR-T cells are described in the art. See, e.g., WO2018/075807. Many other humanized CAR-T switches and complementary CAR-T cells suitable for use in the methods of the invention are also known. See, e.g., WO2018/075807; and Viaud et al., Proc. Natl. Acad Sci. USA 115: E10898-E10906, 2018.

(humanized anti-CD19 light chain variable
region with N-terminal fused GCN4 peptide)
SEQ ID NO: 2
NYHLENEVARLKKLGGGGSDIQMTQSPSSLSASVGDRVTITCRASQ
DISKYLNWYQQKPGKAVKLLIYHTSRLHSGVPSRFSGSGSGTDYTL
TISSLQPEDFATYFCQQGATLPYTFGQGTKLEIK
(humanized anti-CD19 heavy chain variable
region)
SEQ ID NO: 3
QVQLQESGPGLVKPSETLSVTCTVSGVSLPDYGVSWIRQPPGKGLE
WLGVIWGSETTYYNSALKSRLTISKDNSKNQVSLKMSSLTAADTAV
YYCAKHYYYGGSYAMDYWGQGTLVTVSS
(humanized anti-GCN4 light chain variable
region)
SEQ ID NO: 4
QAVVTQEPSLTVSPGGTVTLTCGSSTGAVTTSNYASWVQEKPDHLF
RGLIGGTNNRAPGVPARFSGSLLGGKAALTISGAQPEDEAIYFCVL
WYSDHWVFGGGTKLTVDG
(humanized anti-GCN4 heavy chain variable
region)
SEQ ID NO: 5
QVQLQQSGPGLVKPSETLSITCTVSGFLLTDYGVNWVRQPPGKGLE
WLGVIWGDGITDYNPSLKSRLTVSKDTSKNQVSLKMSSLTDADTAR
YYCVTGLFDYWGQGTTLTVSS
(full sequence of humanized CAR)
SEQ ID NO: 6
QAVVTQEPSLTVSPGGTVTLTCGSSTGAVTTSNYASWVQEKPDHLF
RGLIGGTNNRAPGVPARFSGSLLGGKAALTISGAQPEDEAIYFCVL
WYSDHWVFGGGTKLTVDGGGGGSGGGGSGGGGSGGGGSQVQLQQSG
PGLVKPSETLSITCTVSGFLLTDYGVNWVRQPPGKGLEWLGVIWGD
GITDYNPSLKSRLTVSKDTSKNQVSLKMSSLTDADTARYYCVTGLF
DYWGQGTTLTVSSESKYGPPCPPCPDFWVLVVVGGVLACYSLLVTV
AFIIFWVRSKRSRLLHSDYMNMTPRRPGPTRKHYQPYAPPRDFAAY
RSKRGRKKLLYIFKQPFMRPVQTTQEEDGCSCRFPEEEEGGCELRV
KFSRSADAPAYKQGQNQLYNELNLGRREEYDVLDKRRGRDPEMGGK
PRRKNPQEGLYNELQKDKMAEAYSEIGMKGERRRGKGHDGLYQGLS
TATKDTYDALHMQALPPR
(humanized anti-GCN4 scFv)
SEQ ID NO: 7
QAVVTQEPSLTVSPGGTVTLTCGSSTGAVTTSNYASWVQEKPDHLF
RGLIGGTNNRAPGVPARFSGSLLGGKAALTISGAQPEDEAIYFCVL
WYSDHWVFGGGTKLTVDGGGGGSGGGGSGGGGSGGGGSQVQLQQSG
PGLVKPSETLSITCTVSGFLLTDYGVNWVRQPPGKGLEWLGVIWGD
GITDYNPSLKSRLTVSKDTSKNQVSLKMSSLTDADTARYYCVTGLF
DYWGQGTTLTVSS
(hinge of humanized CAR)
SEQ ID NO: 8
ESKYGPPCPPCPD
(transmembrane domain of humanized CAR)
SEQ ID NO: 9
FWVLVVVGGVLACYSLLVTVAFIIFWV
(CD28 domain of intracellular domain of
humanized CAR)
SEQ ID NO: 10
RSKRSRLLHSDYMNMTPRRPGPTRKHYQPYAPPRDFAAYRS
(4-1 BB domain of intracellular domain of
humanized CAR)
SEQ ID NO: 11
KRGRKKLLYIFKQPFMRPVQTTQEEDGCSCRFPEEEEGGCEL
(CD3-zeta domain of intracellular domain of
humanized CAR)
SEQ ID NO: 12
RVKFSRSADAPAYKQGQNQLYNELNLGRREEYDVLDKRRGRDPEMG
GKPRRKNPQEGLYNELQKDKMAEAYSEIGMKGERRRGKGHDGLYQG
LSTATKDTYDALHMQALPPR
(full light chain sequence of CAR-T switch Fab
SWI019)
SEQ ID NO: 15
NYHLENEVARLKKLGGGGSDIQMTQSPSSLSASVGDRVTITCRASQ
DISKYLNWYQQKPGKAVKLLIYHTSRLHSGVPSRFSGSGSGTDYTL
TISSLQPEDFATYFCQQGATLPYTFGQGTKLEIKRTVAAPSVFIFP
PSDEQLKSGTASVVCLLNNFYPREAKVQWKVDNALQSGNSQESVTE
QDSKDSTYSLSSTLTLSKADYEKHKVYACEVTHQGLSSPVTKSFNR
GEC
(full heavy chain sequence of CAR-T switch Fab
SWI019)
SEQ ID NO: 16
QVQLQESGPGLVKPSETLSVTCTVSGVSLPDYGVSWIRQPPGKGLE
WLGVIWGSETTYYNSALKSRLTISKDNSKNQVSLKMSSLTAADTAV
YYCAKHYYYGGSYAMDYWGQGTLVTVSSASTKGPSVFPLAPSSKST
SGGTAALGCLVKDYFPEPVTVSWNSGALTSGVHTFPAVLQSSGLYS
LSSVVTVPSSSLGTQTYICNVNHKPSNTKVDKKVEPKSC

### III. Human Cancers to Be Treated

The methods of the invention are directed to treating human cancers by directing the sCAR-T cells to tumor cells in a human subject bearing one or more specific target molecules (e.g., CD19) that are recognized by the co-administered CAR-T switches. The switches may interact with a plurality of target cells that express the same (e.g., CD19) or different target molecules (e.g., CD 19 and CD20). In some methods, the cancer cell surface molecule to be targeted by the sCAR-T platform of the invention is a receptor. The receptor may be an extracellular receptor. The receptor may be a cell surface receptor. By way of non-limiting example, the receptor may bind a hormone, a neurotransmitter, a cytokine, a growth factor or a cell recognition molecule. The receptor may be a transmembrane receptor. The receptor may be an enzyme-linked receptor. The receptor may be a G-protein couple receptor (GPCR). The receptor may be a growth factor receptor. The cell surface molecule may be a non-receptor cell surface protein. The target molecule may be a cluster of differentiation proteins. By way of non-limiting example, the cell surface molecule may be selected from CD19, CD20, CD34, CD31, CD117, CD45, CD11b, CD15, CD24, CD114, CD182, CD14, CD11a, CD91, CD16, CD3, CD4, CD25, CD8, CD38, CD22, CD61, CD56, CD30, CD13, CLL1, CD33, CD123, or fragments or homologs thereof. In some preferred embodiments, the target molecule is CD19 as exemplified herein.

Various human cancers can be treated with the methods of the invention. In some embodiments, the cancer to be treated is heterogeneous. In some embodiments, the cancer to be treated is a blood cell malignancy. For example, the cancer to be treated can be derived from bone marrow cells or other blood cells. In these embodiments, the cancer can be derived from a B cell, a T cell, a monocyte, a thrombocyte, a leukocyte, a neutrophil, an eosinophil, a basophil, a lymphocyte, a hematopoietic stem cell or an endothelial cell progenitor. In some embodiments, the cancer to be treated is a relapsed, refractory B cell malignancies. In some preferred embodiments, the cancer to be treated is derived from a CD19-positive B lymphocyte. In some embodiments, the cancer may be derived from a stem cell. For example, the targeting cancer cell may be derived from a pluripotent cell. In some embodiments, the cancer cell to be targeted can be derived from one or more endocrine glands. The endocrine gland may be a lymph gland, pituitary gland, thyroid gland, parathyroid gland, pancreas, gonad or pineal gland.

In some preferred embodiments, methods of the invention are directed to treating subjects afflicted with relapsed/refractory B cell malignancies who have previously received treatment. Such diseases and conditions are well known in the art. See, e.g., Swerdlow et al., WHO classification of tumours of haematopoietic and lymphoid tissues. In: World Health Organization Classification of Tumours, Lyon, France: IARC, Revised 4th Edition (2017); and Swerdlow et al., Blood 2016, 127(20):2375-90. In various embodiments, these subjects can be ones suffering from various forms of B cell lymphoma, including unclassifiable, with features intermediate between diffuse large B cell lymphoma (DLBCL) and classical Hodgkin lymphoma. These include, e.g., DLBCL, not otherwise specified (NOS), DLBCL associated with chronic inflammation, Germinal center B cell type, Activated B cell type, Chronic lymphocytic leukemia (CLL)/small lymphocytic lymphoma (SLL), Transformation of follicular lymphoma (FL) or other indolent histologies to DLBCL, CLL with Richter's transformation, Splenic marginal zone lymphoma, Splenic B cell lymphoma/leukemia, unclassifiable (Splenic diffuse red pulp small B cell lymphoma, and Hairy cell leukemia-variant), Extranodal marginal zone lymphoma of mucosa-associated lymphoid tissue (MALT lymphoma), Nodal marginal zone lymphoma, Mantle cell lymphoma (MCL) including In situ mantle cell neoplasia, Follicular lymphoma (FL) (including In situ follicular neoplasia and Duodenal-type follicular lymphoma), Large B cell lymphoma with IRF4 rearrangement, Primary cutaneous follicle center lymphoma, and Epstein Bar Virus (EBV)+DLBCL (NOS) (including EBV+DLBCL). Suitable tumors to be treated with methods of the invention also include various primary mediastinal (thymic) large B cell lymphoma. These include, e.g., ALK+ large B cell lymphoma, HHV8+DLBCL (NOS), High-grade B cell lymphoma, with MYC and BCL2 and/or BCL6 rearrangements, High-grade B cell lymphoma (NOS), Burkitt lymphoma, and Lymphoplasmacytic lymphoma (LPL) (including Waldenström macroglobulinemia (WM)).

Some methods of the invention are directed to treating leukemia, e.g., CD-19 positive leukemia. Specific examples of leukemias include myelogenous leukemia, lymphoblastic leukemia, myeloid leukemia, an acute myeloid leukemia, myelomonocytic leukemia, neutrophilic leukemia, myelodysplastic syndrome, B-cell lymphoma, Burkitt lymphoma, large cell lymphoma, mixed cell lymphoma, follicular lymphoma, mantle cell lymphoma, Hodgkin lymphoma, recurrent small lymphocytic lymphoma, hairy cell leukemia, multiple myeloma, basophilic leukemia, eosinophilic leukemia, megakaryoblastic leukemia, monoblastic leukemia, monocytic leukemia, erythroleukemia, erythroid leukemia and hepatocellular carcinoma. In some embodiments, the disorder to be treated is a hematological malignancy. In some embodiments, the disorder to be treated is a B cell malignancy. In some embodiments, the disorder to be treated is a chronic lymphocytic leukemia. In some embodiments, the disorder to be treated is an acute lymphoblastic leukemia. In some embodiments, the disorder to be treated is a CD19-positive Burkitt's lymphoma.

In some preferred embodiments, the cancer to be treated is a CD19-positive tumor or malignancy. In some of these embodiments, the cancer to be treated is a B cell cancer or B cell malignancy. B cell cancer or B cell malignancy encompass B-cell lymphomas which account for a major portion of non-Hodgkin lymphomas (NHL). Examples of these B cell cancers include, e.g., diffuse large B-cell lymphoma (DLBCL), follicular lymphoma, chronic lymphocytic leukemia (CLL), small lymphocytic lymphoma (SLL), mantle cell lymphoma (MCL), marginal zone lymphoma, Burkitt lymphoma, lymphoplasmacytic lymphoma (Waldenstrom macroglobulinemia), hairy cell leukemia (HCL), primary central nervous system (CNS) lymphoma, and primary intraocular lymphoma.

### IV. Administration and Dosages

In the practice of the human cancer therapies described herein, subjects in need of treatment are administered a pharmaceutical composition that contains a therapeutically effective amount of the sCAR-T platform described herein. As described herein, the therapeutically effective amount should also be optimal so that subjects receiving the dosage regimen will not experience unacceptable toxicity. In addition to the sCAR-T platform, the composition can contain one or more pharmaceutically acceptable carrier or agent, e.g., salts, excipients or vehicles. The pharmaceutically acceptable carrier can be any suitable pharmaceutically acceptable carrier. It can be one or more compatible solid or liquid fillers, diluents, other excipients, or encapsulating substances which are suitable for administration into a human or veterinary patient (e.g., a physiologically acceptable carrier or a pharmacologically acceptable carrier). The administration of the sCAR-T platform may be in any order. For example, in some embodiments, the sCAR-T cells may be administered simultaneously with CAR-T switch administration. In some other methods, the CAR-T switch may be administered prior to sCAR-T cell administration. In some preferred embodiments, the sCAR-T cells are administered prior to CAR-T switch administration, as exemplified herein.

General guidance for preparation and administration of the therapeutic compositions of the invention are described in the art. See, e.g., Goodman & Gilman's The Pharmacological Bases of Therapeutics, Hardman et al., eds., McGraw-Hill Professional (10^{th} ed., 2001); Remington: The Science and Practice of Pharmacy, Gennaro, ed., Lippincott Williams & Wilkins (20^{th} ed., 2003); and Pharmaceutical Dosage Forms and Drug Delivery Systems, Ansel et al. (eds.), Lippincott Williams & Wilkins (7^{th} ed., 1999). Methods of administering therapeutic compositions to a subject can be accomplished based on procedures routinely practiced in the art. See, e.g., Remington: The Science and Practice of Pharmacy, Mack Publishing Co., 20^{th} ed., 2000; Iwasaki et al., Jpn. J. Cancer Res. 88:861-6, 1997; Jespersen et al., Eur. Heart J. 11:269-74, 1990; and Martens, Resuscitation 27:177, 1994. Typically, the sCAR-T platform-containing composition is administered (e.g., via injection) in a physiologically tolerable medium, such as phosphate buffered saline (PBS). The therapeutic compositions can be administered to subjects in need of treatment via any appropriate route. In some preferred embodiments, the composition is administered to the subject via parenteral administration. In these embodiments, the administered composition should contain a sterile aqueous preparation that is preferably isotonic with the blood of the recipient.

The CAR-T switch and the complementary sCAR-T cells should be administered to the subject in a sufficient amount to inhibit the growth and promote the regression of a tumor in the subject. The sufficient number of cells can be determined based on the type of tumor, its size and stage of development, the route of administration, the age of the specific subject to be treated and other factors that will be readily apparent to one of ordinary skill in the art of treating tumors. As a general guidance for administration via injection or implantation, the subject to be treated is administered with one sCAR-T cell dose containing from about 1×10⁵ to about 1×10⁸ CAR⁺ cells per kg of body weight. While the one dose of the sCAR-T cells is preferably administered to the subjects in a single administration, splitting the one dose into multiple (e.g., 2, 3, or 4) administrations is also suitable in the practice of the invention. In some embodiments, the dose can contain from about 5×10⁶ to about 2×10⁷ cells per kg of body weight. In various embodiments, a sCAR-T cell dose can contain about 2.5×10⁵, 5×10⁵, 7.5×10⁵, 1×10⁶, 2.5×10⁶, 5×10⁶, 7.5×10⁶, 1×10⁷, 2.5×10⁷, 5×10⁷, 7.5×10⁷ or more cells per kg of body weight. Alternatively, for subjects with normal body weight (e.g., around 70 kg, or from about 50 kg to about 90 kg), a flat dose of the sCAR-T cell can be administered. In various embodiments, the flat CAR⁺ T cell dose can be about 20×10⁶, 40×10⁶, 60×10⁶, 80×10⁶, 100×10⁶, 120×10⁶, 140×10⁶, 160×10⁶, 180×10⁶, 200×10⁶, 300×10⁶, 400×10⁶, 500×10⁶, 600×10⁶, 700×10⁶, 800×10⁶, 900×10⁶, 1000×10⁶ or more cells.

In some embodiments, the subject can be administered with a flat dose of about 35×10⁶ to about 700×10⁶ (or about 1400×10⁶) CAR⁺ T cells. For a patient with a 70 kg body weight, this CAR⁺ T cell dose is equivalent to about 0.05×10⁷ to about 1×10⁷ (or 2×10⁷) cells/kg body weight. In some of these embodiments, the subject is administered with a flat dose of about 140×10⁶ cells to about 700×10⁶ cells as exemplified herein. For a patient with a 70 kg body weight, this CAR-T cell dose is equivalent to about 0.2×10⁷ to 1×10⁷ cells/kg body weight. For treating subjects with substantially different weight, the CAR-T cell dose based on per kg body weight can be accordingly adjusted. In some preferred embodiments, the subject is administered with a flat dose of about 140×10⁶ cells, which is equivalent to a 0.2×10⁷ cells/kg body weight for subjects weighing about 70 kg.

For the CAR-T switch molecule, a daily dose can be in the range between about 0.0001 mg to about 10 mg per kg of body weight. In some embodiments, the daily dose of the switch to be administered is between about 0.00025 mg to about 2.5 mg per kg of body weight. In some embodiments, the daily dose of the switch to be administered is any amount from about 0.0005 mg (or 0.00075 or 0.001 mg) to about 0.5 mg (or 1 mg, 1.5 mg or 2 mg) per kg of body weight. In some embodiments, the daily dose of the switch to be administered is any amount from about 0.01 mg (or 0.005 mg) to about 0.1 mg or 1 mg per kg of body weight. In some embodiments, the daily dose of the switch to be administered is any amount from about 0.045 mg to about 0.075 mg per kg of body weight. In various embodiments, a daily dose of the CAR-T switch molecule to be administered can be about 0.0001 mg, 0.00025 mg, 0.0005 mg, 0.00075 mg, 0.001 mg, 0.0025 mg, 0.005 mg, 0.0075 mg, 0.01 mg, 0.025 mg, 0.05 mg, 0.06 mg, 0.075 mg, 0.1 mg, 0.25 mg, 0.5 mg, 0.75 mg, 1 mg, 1.5 mg, 2 mg, 2.5 mg, 3 mg, 4 mg, 5 mg or more per kg of body weight. In some embodiments, a daily dose of the CAR-T switch molecule to be administered can be about 0.01 mg, 0.025 mg, 0.03 mg, 0.04 mg, 0.045 mg, 0.05 mg, 0.055 mg, 0.06 mg, 0.065 mg, 0.070 mg, 0.075, 0.085, or 0.095 mg. In some preferred embodiments, a daily dose of the CAR-T switch molecule to be administered is about 0.06 mg. As with the administration of the CAR-T cells, the noted doses the CAR-T switch molecule can also be administered in a single administration or by multiple administrations.

In some embodiments, the CAR-T cells are administered to the subject to be treated only at the beginning of the treatment (e.g., once or twice administration), while the CAR-T switch molecule is administered multiple times based on an “on/off schedule” during the course of the treatment, e.g., throughout the first few weeks or months thereafter. As exemplified herein, the on/off schedule comprises multiple cycles, with each cycle containing an “on” phase and an “off” phase. The CAR-T switch is administered to the subject during the “on” phase, while no CAR-T switch is administered during the “off” phase. In various embodiments, the subject can be administered the switch molecule twice a day, daily, every other day, every three days or longer during an “on” phase, which lasts a specific period of time (e.g., any length from about one day to a few weeks). The “off” phase of the cycle can be any period that is longer than the interval of the administration during the “on” phase. Thus, for example, when the administration takes place every day or every other day during the “on” phase, the “off” phase can last any length of time from about a few days to about a few years. In various embodiments, the “on” phase can be, e.g., 1 day, 2 days, 3 days, 4 days, 5 days, 6 days, 1 week, 2 weeks, 3 weeks, or longer, with administration taking place once every day, every two days, every 3 days or longer. For each of these “on” phases, the “off” phase can be, e.g., 4 days, 5 days, 6 days, 1 week, 2 weeks, 3 weeks, 4 weeks, 2 months, 3 months, 4 months, 5 months, 6 months, 1 year or longer. The length of time of the “on” phase and the “off” phase can be respectively the same for the multiple cycles. Alternatively, the duration of the “on” phase and/or the “off” phase can vary among the different cycles. As a specific exemplification as detailed below, the treatment can contain at least about 6 cycles. Each cycle can last 28 days, with the on and off phase being 7 days and 21 days, respectively, as exemplified herein.

The number of cells and frequency of administration can also vary depending on whether the treatment is prophylactic or therapeutic. In prophylactic applications, a relatively low number of cells may be administered at relatively infrequent intervals over a long period of time. Some subjects may continue to receive treatment for the rest of their lives. In therapeutic applications, a relatively high number of cells at relatively short intervals may be required until progression of the tumor is reduced or terminated, and preferably until the subject shows partial or complete regression of the tumor. Thereafter, the subject can be administered a prophylactic regime.

In some embodiments, therapeutic compositions described herein can be administered locally via implantation into the affected area of a membrane, sponge, or other appropriate material on to which a sCAR-T platform disclosed herein has been absorbed or encapsulated. Where an implantation device is used, the device may be implanted into any suitable tissue or organ, and delivery of a CAR-T platform disclosed herein may be directly through the device via bolus, or via continuous administration, or via catheter using continuous infusion.

As specific exemplifications, some therapeutic methods of the invention entail first administering (e.g., via infusion) to a subject with CD19-positive relapsed/refractory B cell malignancy one dose of about 0.35×10⁸ to about 7×10⁸ CAR-T cells that contain a CAR sequence set forth in SEQ ID NO:6, and then administering to the subject during one or more infusion cycles a CAR-T switch molecule that is an anti-CD19 Fab antibody containing a light chain variable region and a heavy chain variable region sequence set forth as SEQ ID NOs:2 and 3, respectively. Each of the infusion cycles contains (i) an “on” phase of about 5 to about 9 days and (ii) an “off” phase of about 14 to about 28 days. During the on phase, a daily dose of about 0.045 mg to about 0.075 mg per kg of body weight of the switch is administered. In some of these embodiments, the employed CAR-T cells are autologous to the subject. In some embodiments, the infused dose of the CAR-T cells is about 1.4×10⁸ cells, and the daily dose of the CAR-T switch infused during the “on” phase is about 0.06 mg per kg of body weight. In some embodiments, the “on” phase is about 7 days, and the “off” phase is about 21 days. In various embodiments, the number of infusion cycles for administering the CAR-T switch can be 2, 3, 4, 5, 6 or more. In some embodiments, the subject is given lymphodepletion preconditioning prior to infusion of the CAR-T cells and the switch molecule. In some of these embodiments, the lymphodepletion preconditioning is via chemotherapy with cyclophosphamide and fludarabine.

The sCAR-T platform described herein can be used in combination with other known regimens for treating cancers. Methods for co-administration with an additional therapeutic agent are well known in the art. See, e.g., Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; and Chabner and Longo (eds.) (2001) Cancer Chemotherapy and Biotherapy, Lippincott, Williams & Wilkins, Phila., Pa.

### EXAMPLES

The following examples are provided to further illustrate the invention but not to limit its scope. Other variants of the invention will be readily apparent to one of ordinary skill in the art and are encompassed by the appended claims.

### Example 1. IND-Enabling Studies of sCAR-T Therapy for Human Cancer Patients

A “switchable” CAR-T (sCAR-T) therapy has the activity of the sCAR-T cells is controlled by an antibody-based switch. The switch targets the tumor antigen, and the sCAR recognizes a unique peptide engrafted on the switch. The switch creates a bridge between the sCAR-T cell and the tumor cell, activating the sCAR-T cells and inducing tumor cell killing. The sCAR-T treatment platform employed by the inventors contains a humanized inert CAR-T cell (aka CLBR001 herein) and a corresponding switch molecule (aka SWI019 herein). The extracellular domain of the CAR (SEQ ID NO:6) of the CAR-T cell contains a humanized scFv antibody fragment (SEQ ID NO:7) that specifically binds a GCN4 derivative peptide (SEQ ID NO:1) in the switch molecule. The switch molecule additionally contains a CD19 targeting Fab antibody (with heavy and light chain variable region sequence shown in SEQ ID NOs:3 and 2, respectively) that has its light chain N-terminus fused to the GCN4 peptide. More detailed information about this sCAR-T platform has been reported in, e.g., WO2018/075807. It was demonstrated that, while each of the switch molecule and the sCAR-T cell individually is designed to be inactive, their combination led to complete elimination of tumors in xenograft and syngeneic models. The short half-life of the switch allows for a rapid modulation of sCART-cell activity through the switch dosing. Moreover, by swapping different switches, sCAR-T cells can be modularly redirected against other tumor targets. Further, it was shown that the cyclical on/off stimulation of the sCAR-T cells provides improved memory and persistence of the sCAR-T cells.

Using this exemplified sCAR-T platform, the inventors undertook IND-enabling studies in preparation and support for a first in human (FIH) clinical study of the sCAR-T therapy. The preclinical development of a platform which includes a sCAR-T cell that lacks any endogenous antigen target, in combination with an antibody-based switch molecule that lacks intrinsic activity in the absence of the sCAR-T cell, necessitated development of novel approaches. Fidelity of such a system is essential to control, thus, to confirm CLBR001 cells did not activate in the presence of normal tissues, in vitro activity studies were performed by co-culturing CLBR001 cells, in the presence or absence of SWI019, and a panel of 14 primary cells. This panel represented a survey of vital tissues throughout the body. The results indicate that CLBR001 did not demonstrate activity in any of the 14 cell types tested, supporting a high fidelity of CLBR001 recognition for SWI019.

Because SWI019 lacks activity in the absence of CLBR001 cells, traditional toxicology studies to identify the “no adverse effect level” (NOAEL) in support of the first in human starting dose were not applicable. In such cases, a minimal anticipated biological effect level (MABEL) is commonly used to support starting human dose based on the predicted Cmax. However, the femtomolar-level in vitro activity of SWI019 in combination with CLBR001 resulted in starting doses that were modeled to be far outside of the range of potential clinical activity. Therefore, an in vivo-based approach to determine MABEL was developed. In this study, CLBR001 cells were administered in NSG mice bearing CD19+ Nalm-6 cell tumors and a single dose titration of SWI019 was performed. Comparison of the SWI019 efficacious dose (ED50) values for anti-tumor activity, peripheral cytokines, and CLBR001 cells in peripheral blood demonstrated that reduction in Nalm-6 tumor burden was the most sensitive marker of activity. Extravasation of CLBR001 cells from peripheral blood and all three cytokines (IFN-γ, IL-2, and TNF-α) exhibited weaker ED₅₀ values. Therefore, antitumor activity (Nalm-6 tumor burden reduction) was chosen as the parameter with which to determine the in vivo MABEL. Allometric scaling, using mouse and NHP SWI019 PK data, was used to model a SWI019 recommended dose in humans corresponding to the ED₂₀ of the in vivo MABEL study. It was found that, compared to the dose modeled using the in vitro MABEL approach, the in vivo MABEL approach afforded a first in human starting point which was ˜13000-fold higher. Results from this study provides an excellent starting point for the first in human study which balances safety and the potential for patient benefit.

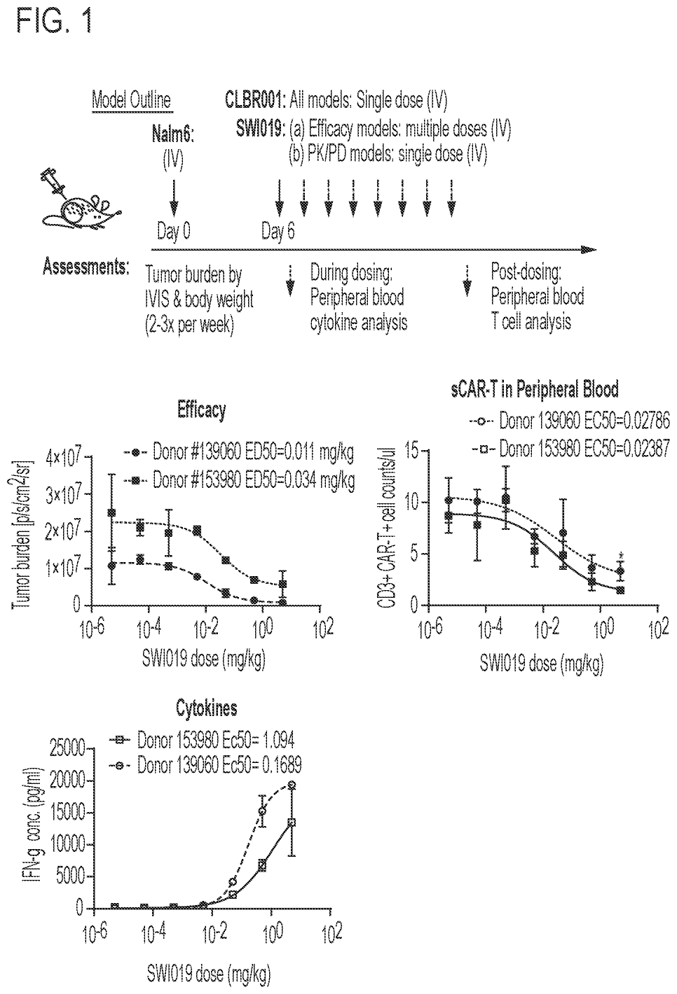

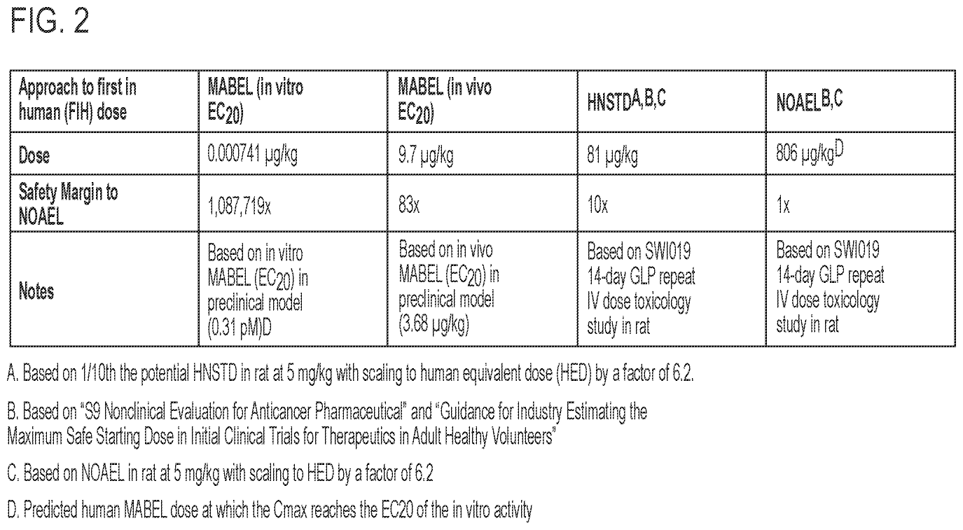

FIGS. 1 and 2 provide some more detailed information related to the studies:

FIG. 1 relates to in vivo MABEL based approach to assess FIH dosage. In vitro MABEL simulations presented challenges to delivery of such low quantities and would potentially require many dose cohorts of patients before a therapeutic dose is reached. Therefore, an in vivo MABEL approach was developed with Nalm-6 xenograft. Tumor burden reduction, cytokines and sCAR-T in peripheral blood were examined in these studies.

FIG. 2 summarizes simulated human equivalent doses of the SWI019 switch. They are based on the most sensitive markers monitored in the vitro and in vivo pharmacological analysis in the treatment with the combination of the CLBR001 CAR-T cell and the SWI019 switch.

In conclusion, the in vivo MABEL approach allowed the determination of a first in human (FIH) starting switch dose (9.7 μg/kg) that is about 13,000-fold higher compared to the in vitro MABEL simulation. This approach provides an excellent starting point for the first in human study which balances safety and the potential for patient benefit.

### Example 2. Human Study of Switchable CAR-T Cell Platform for Treating Tumors

Based on the results from the IND-enabling animal studies described above, a human clinical trial (NCT04450069) of the on/off sCAR-T therapy (CLBR001+SWI019) for treating relapse/refractory B cell malignancies was initiated. CD19-targeting chimeric antigen receptor (CAR) T cells are a transformative treatment option for patients with relapsed, refractory B cell malignancies. Despite remarkable responses in heavily pretreated patients, challenges remain related to toxicities including cytokine release syndrome (CRS) and immune effector cell-associated neurotoxicity syndrome (ICANS), along with relapse related to tumor antigen loss. The sCAR-T treatment described herein is intended to address these challenges.

The Phase 1, open-label, dose escalating study was intended to evaluate the safety, tolerability, pharmacokinetics, pharmacodynamics and clinical activity of the combination of CLBR001 and SWI019 in human patients with relapsed/refractory B-cell malignancies. Specifically, autologous CLBR001 sCAR-T cells were manufactured from patient-derived apheresis material at a centralized manufacturing facility. Following cyclophosphamide and fludarabine lymphodepletion, patients received a single dose of CLBR001 cells, followed by daily infusion of SWI019 for 7 days. The CAR-T cell dose is from about 140×10⁶ cells to about 700×10⁶ cells. For a patient with a 70 kg body weight, this single CAR-T cell dose is equivalent to about 0.2×10⁷ to 1×10⁷ cells/kg body weight. The SWI019 switch molecule was administered on a 28-day cycle for up to 6 cycles. Each cycle consists of daily IV administration for 7 days and then 21 days without dosing. Dose escalation of CLBR001 and SWI019 was determined in the initial cohorts by a [3+3] design followed by implementation of Bayesian adaptive design (BAYDE) decision rules.

Three patients (2 follicular lymphoma and 1 mantle cell lymphoma) in cohort 1 (140×10⁶ CAR-T cells+10 μg/kg SWI019 switch) have evaluable safety and response data as of the data cut-off. CLBR001+SWI019 was well tolerated with no DLTs observed in cohort 1. CLBR001 cell infusion was well tolerated with no adverse events attributable to the cell product in any patients during the observation period prior to SWI019 dosage, indicating CLBR001 cells do not have activity in the absence of SWI019. Elevated serum cytokine levels and CLBR001 expansion in peripheral blood was observed only after SWI019 administration. SWI019 dosage was well tolerated with 1 case of concomitant Grade 1 CRS and Grade 2 ICANS that occurred in cycle 2. This event subsided within 24 hours of administration of dexamethasone with no recurrence of CRS observed with continued administration of a reduced dose (50%) of SWI019, providing support for the tunability of the platform. In the first cohort, 2 of 3 patients experienced a complete response by Lugano criteria.

This is the first report of a switchable CAR-T cell platform in patients with B cell malignancies. The combination of CLBR001+SWI019 was found to be safe and well tolerated in patients with B cell malignancies with encouraging clinical activity in cohort 1 using the lowest doses of both CLBR001 and SWI019.

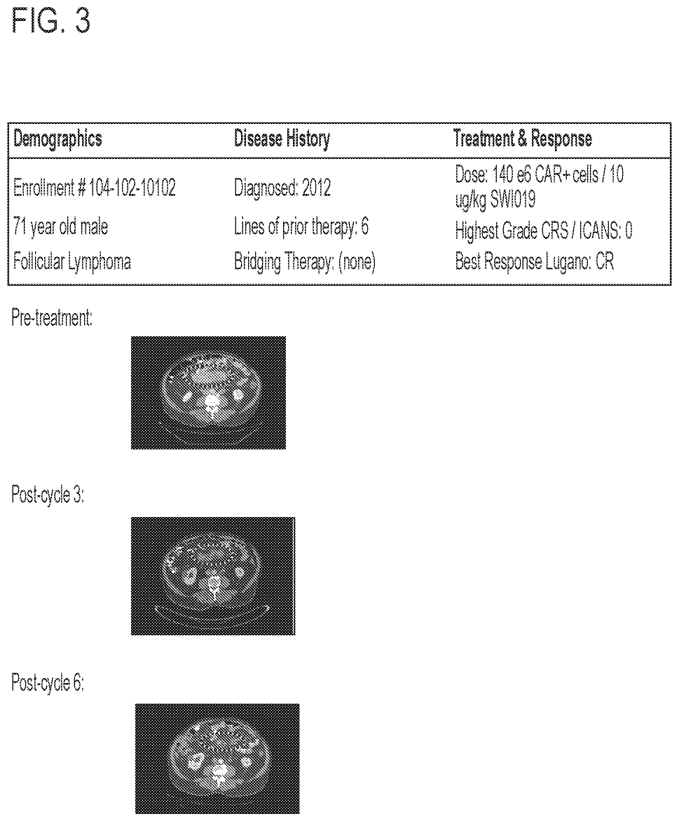

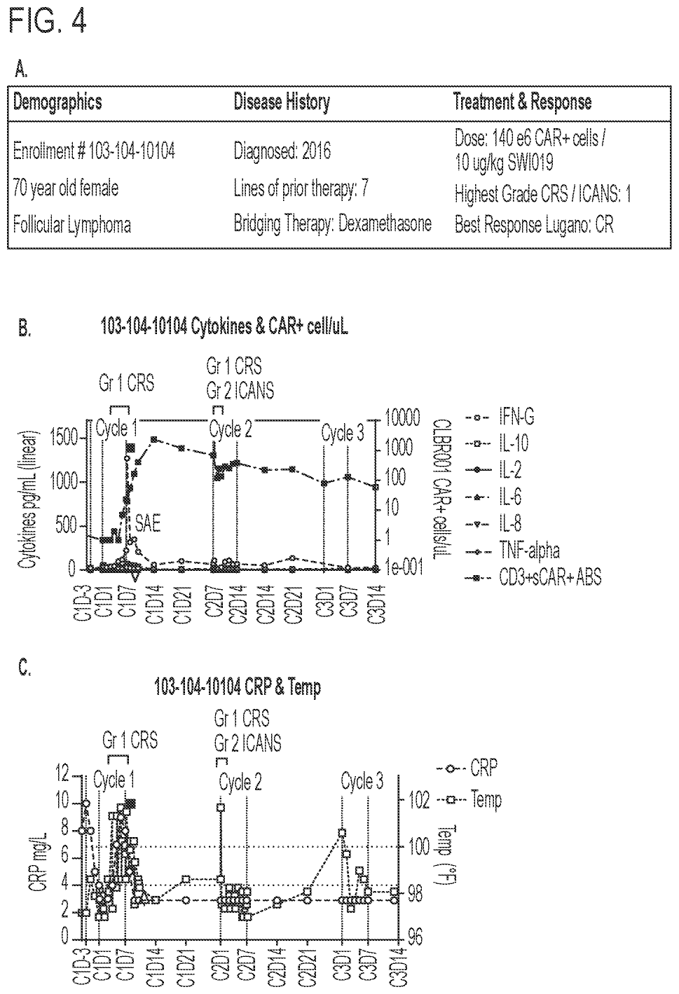

Some detailed information about the design and results of the human therapy is provided in FIGS. 3 and 4:

FIG. 3 shows effect of treatment in one patient as indicated by CT scan before treatment, post treatment Cycle 3 and post treatment Cycle 6. Patient demographics, disease history and treatment response are summarized at the top. The 3 CT scan images at the bottom show mesenteric lymph node target lesion at the 3 time points, respectively. The results indicate that, at baseline (“pre-treatment”), there were 5 target lesions in the mesenteric, inguinal, iliac and para-aortic lymph nodes. Mesenteric lymph node mass was measuring 14.1 cm. The overall Deauville 5PS score by PET was 4. After treatment, there is a decrease in all 5 FDG-avid target lesions. No new target masses were observed, and there is no evidence of lymphoma in bone marrow by IHC. The patient had a continuing complete response (CR) by Lugano and RECIL at 11 months after treatment started.

FIG. 4 shows rapid resolution of toxicity in another treated patient. Patient demographics, disease history and treatment response are summarized in Panel A. Panel B shows that cessation of switch dosing at Day 7 of Cycle 1 (C1D7) led to rapid resolution of fever despite continued expansion of CAR-T, demonstrating functional “off” switch capabilities. Panel C shows that Grade 1 Cytokine Release Syndrome (Gr 1 CRS)/Grade 1 Immune effector Cell Associated Neurotoxicities (Gr 1 ICANs) on Cycle 2 Day 1 dose of SWI019 rapidly resolved with dex, and that continued dosing at 50% dose (5 μg/kg) did not elicit further toxicity.

The results from the human clinical trial indicate that the combination of CLBR001 sCAR-T cell+SWI019 switch molecule is safe and well-tolerated. The combination shows encouraging signs of clinical activity in patients with B cell malignancies. To summarize, there were no adverse events related to CLBR001 prior to SWI019 dosing, demonstrating the safety of the CLBR001 cells alone and fidelity of the CBLR001+SWI019 interaction (i.e., CLBR001 does not react with endogenous human targets). Two CRs were achieved with a maximum grade CRS/ICANS=1 at the lowest doses of CLBR001 and SWI019 (Cohort 1; 140×10⁶ CAR-T cells, 10 μg/kg SWI019). Reactivation of CLBR001 by SWI019 in cycle ≥2 demonstrates functional reversibility of the platform. Rapid resolution of toxicity was achieved by holding or reducing SWI019 dose level, demonstrating potential for greater safety with the CLBR001+SWI019 platform

### Example 3. Clinical Studies to Identify Recommended Phase II Dose (RP2D)

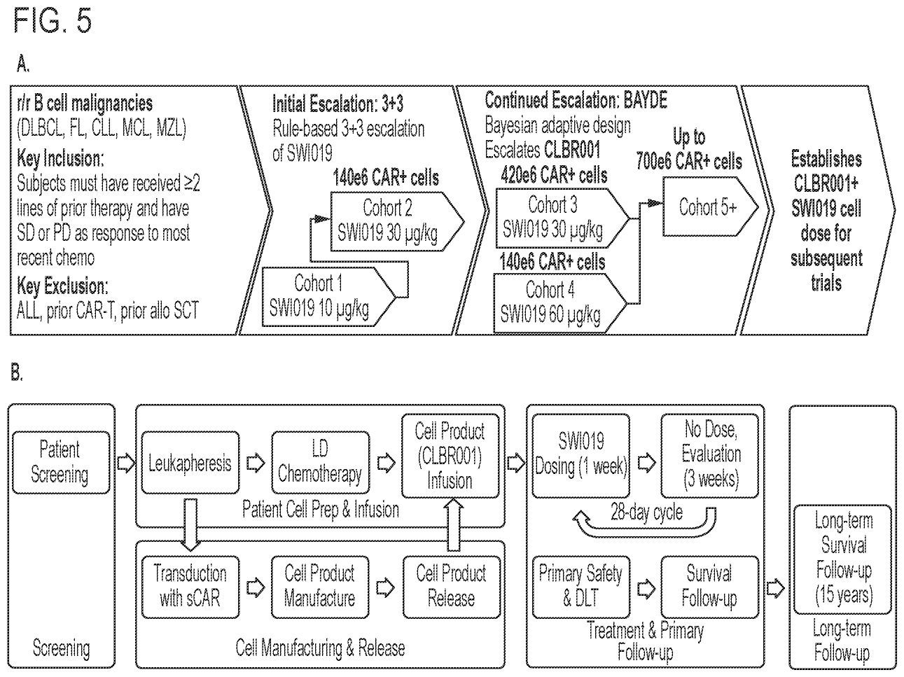

A dose escalation Phase I human clinical trial was conducted to examine optimal dosage regimen of the CLBR001+SWI019 switchable CAR-T treatment of subjects with B cell malignancies (CBR-sCAR19-3001, NCT04450069), as illustrated in FIG. 5. FIG. 5A provides an overview of the design of the Phase I human studies. Subjects eligibility included relapsed/refractory B cell malignancies including diffuse large B cell lymphoma (DLBCL), Follicular Lymphoma (FL), Chronic Lymphocytic Leukemia (CLL), Mantle Cell Lymphoma (MCL), Marginal Zone Lymphoma (MZL), and other Histologies. Subjects with acute lymphoblastic leukemia (ALL), those that had prior CAR-T cell therapy, and those with prior allogeneic stem cell transplant were excluded. The first two cohorts of the study tested dose escalation of the switch (SWI019) starting at 10 μg/kg and proceeding to 30 μg/kg in a 3+3 dose escalation design. Subjects in the first two cohorts received 140e6 CAR+ cells prior to receiving SWI019. The next cohorts used a Bayesian adaptive dose escalation (BAYDE) design to determine optimal dose. Cohort 4 assessed 60 μg/kg of SWI019 with 140e6 CAR+ cells, and Cohort 5 assessed 30 μg/kg of SWI019 with 420e6 CAR+ cells. Cohort 5 could optionally test doses determined by the BAYDE model. The goal of the design was to establish the recommended Phase II CAR+ cell and SWI019 dose for use in future studies. The established CLBR001 cell dose could be used with SWI019 or in combination with other switches in future studies.

As indicated in the treatment schedule overview shown in FIG. 5B, subjects enrolled in the CBR-sCAR19-3001 trial were treated as follows. Subjects were first requested to provide consent and then screened for eligibility. Those subjects determined to meet the predefined inclusion and exclusion criteria had leukapheresis product shipped to a centralized manufacturing facility where T cells were isolated and transduced with the sCAR vector. The cell product was manufactured and released, followed by shipment back to the subject treatment site. The subject received lymphodepleting chemotherapy (typically cyclophosphamide and fludarabine), followed by infusion of CLBR001 cells. Subjects were then treated with a cycle of SWI019 as follows: 7 daily doses of SWI019 followed by 21 days of no dosing in a 28 day cycle. Subjects were evaluated for dose limiting toxicities, responses (by PET/CT every 28 days as appropriate), and pharmacodynamic biomarkers (cytokines in peripheral blood), among other assessments.

TABLE 1
Summary of outcomes from first 9 subjects with available data:

| | sCAR19-3001 | ZUMA-1 | JULIET | TRANSCEND |
|---|---|---|---|---|
| | CLBR001 + SWI019 | Yescarta | Kymriah | Breyanzi |
| | Calibr¹ | Kite² | Novartis² | BMS² |
| ORR | 7/9 (78%) | 74% | 52% | 73% |
| CR | 6/9 (67%) | 54% | 40% | 53% |
| CRS (Grade ≥ 3) | 2/9 (22%) | 10% | 22% | 2% |
| Median time to resolution | 1 day | 8 days | 7 days | 5 days |
| CRS (any grade) | | | | |
| ICANS (Grade ≥ 3) | 0% | 32% | 11% | 10% |
| Median time to resolution | 3 days | 17 days | 14 days | 11 days |
| ICANS (any grade) | | | | |
| TLS (Grade ≥ 3) | 0% | 1% | 1% | 1% |
| Treatment-Related Deaths | 0% | 2% | — | 1% |
| Infections (Grade ≥ 3) | 3/9 (33%) | 28% | 20% | 12% |
| % Subjects rcv'd bridging | 5/9 (56%) | 0% (Not | 90% | 59% |
| therapy | | permitted) | | |
| Tocilizumab usage | 3/9 (33%) | 43% | 14% | 20% |

Source of ZUMA-1, JULIET and TRANSCEND: Westin et al., Hematol. 96: 1295-1312, 2021

In the first 9 subjects with available data as of the data cutoff, 7 had a partial response (PR) or complete response (CR) (78%) and 6 had a CR (67%). Two of the 9 subjects had Grade 3 or higher cytokine release syndrome (CRS) and none of the subjects had grade 3 or higher immune effector cell associated neurotoxicity (ICANS). Among the first 9 subjects, the median time to resolution of any grade CRS was 1 day, and the median time to resolution of any grade ICANS was 3 days. This compares favorably with the median time to resolution of CRS or ICANS for three FDA approved CAR-T cells products. The reduction in the duration of CRS is expected to be due to the ability to withhold or reduce SWI019 dosing which is not possible with conventional CAR-T cell products.

Outcomes from the first 14 subjects in the first 4 cohorts treated in the CBR-sCAR19-3001 trial are shown in Table 2. No dose limiting toxicities (DLT) occurred in the first 3 cohorts and 2 DLTs occurred in cohort 4. DLTs were defined as treatment related adverse events occurring during the DLT period which was day 35 post first dose of SWI019 for cohorts 1 and 2 and day 28 post first dose SWI019 for cohorts 3 and 4. Late onset DLT response was defined as treatment related adverse events meeting DLT criteria that occurred after the DLT windows. Efficacy response was defined as subjects experiencing PR or CR. As of the data cut off, two subjects were pending and one subject was unknown. Pharmacodynamic response (PD) was defined as subjects with peripheral blood cytokine levels that increased 3× over baseline post SWI019 dosing.

TABLE 2
Listing of the first 14 subjects

| Dose Cohort | | DLT | Late Onset | Efficacy | PD |
|---|---|---|---|---|---|
| (SWI019/CLBR001) | Subject | Response | DLT Response | Response | Response |
| Cohort 1 | 102-001-10101 | No | No | No | Yes |
| (10 μg/kg/140e6) | 104-102-10102 | No | No | Yes | Yes |
| | 103-104-10104 | No | No | Yes | Yes |
| Cohort 2 | 106-105-10201 | No | No | No | Yes |
| (30 μg/kg/140e6) | 109-108-10202 | No | Yes | Yes | Yes |
| | 107-111-10203 | No | No | Yes | Yes |
| | 104-113-10301 | No | No | Yes | Yes |
| Cohort 3 | 108-116-10402 | No | No | Yes | Yes |
| (60 μg/kg/140e6) | 107-120-10302 | No | No | No | Yes |
| | 105-123-10407 | No | No | Pending | Yes |
| | | | | (Yes) | |
| Cohort 4 | 105-118-10403 | No | No | Yes | Yes |
| (30 μg/kg/420e6) | 107-119-10404 | No | No | Unknown | Yes |
| | | | | (No) | |
| | 107-121-10405 | Yes | No | Pending | Yes |
| | | | | (Yes) | |
| | 104-122-10406 | Yes | No | Yes | Yes |

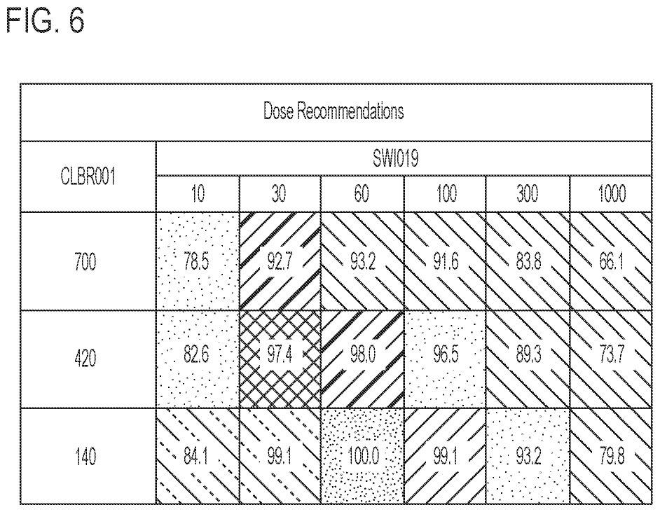

To determine the optimal switch and CLBR001 dose (OSD), a BAYDE model was employed using the data from the table above (FIG. 6). BAYDE is a dose finding design using dose-toxicity/response relationship curves based on historical and cumulative study data to recommend safe, active and effective dosing regimens via Bayesian Adaptive Modeling and Bayesian Logistic Regression Models (BRLM). Recommended doses are optimally selected across dose ranges. The BAYDE approach supports better decision making using initial dose relationships based on historical safety, toxicity, PD, and efficacy data, well-established Bayesian modelling approaches, and dose relationships updated during the trial. The dose selection goals are to: (a) provide optimal dosing recommendations using available data (b) identify a maximum tolerated dose (MTD) or (c) identify OSD levels, that are lower than the MTD and have acceptable PD and/or efficacy response. Doses levels (a) are safe when there is less than a 25% risk (safety acceptance threshold) that the DLT rate exceeds 33% and (b) have an acceptable PD and/or efficacy response when the estimated response rate is at least 50%. For the BAYDE model in this trial, four endpoints were used for dose selection: 1: Binary variable for standard DLT, 2: Binary variable for late-onset DLT, 3: Binary variable for efficacy response, 4: Binary variable for PD response. Allowable dose ranges were SWI019 10-1000 μg/kg and CLBR001 140-700e6 CAR+ cells.

To compute the clinical utility index for ranking combinations of CLBR001 and SWI019 doses, cumulative subject data was entered into the model, response models were updated, and posterior probabilities were estimated using Bayesian methods and simulations. The clinical utility index shown in FIG. 6 is optimal at 100, indicating an appropriate balance of efficacy and potential for toxicity. The above clinical utility index assumed pending subjects were “yes” for efficacy and unknown subject was “no” for efficacy. Thus the optimal switch and CLBR001 dose chosen for expansion was 60 μg/kg of SWI019 and 140e6 CAR+ cells.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

We claim:

1. A method of treating, arresting growth of, and/or promoting regression of, a CD19-positive malignancy in a human subject, comprising administering to the subject (a) a chimeric antigen receptor-T cell switch molecule (CAR-T switch) comprising an anti-CD19 Fab antibody that comprises a light chain variable region and a heavy chain variable region sequence set forth as SEQ ID NOs:2 and 3, respectively, and (b) a complementary CAR-T cell comprising a CAR sequence set forth in SEQ ID NO:6; thereby treating, arresting growth of, and/or promoting regression of, the B cell malignancy in the subject.

2. The method of claim 1, wherein the anti-CD19 Fab antibody comprises a light chain sequence and a heavy chain sequence set forth as SEQ ID NOs:15 and 16, respectively

3. The method of claim 1, wherein the subject is administered with one dose of the CAR-T cell at the beginning of the treatment, and multiple doses of the CAR-T switch during the course of the treatment.

4. The method of claim 3, wherein the subject is infused with one dose of the CAR-T cells, followed by one or multiple cycles of infusion of the CAR-T switch; wherein each cycle comprises an “on” phase of daily infusion of the CAR-T switch for about 5 to about 9 days, and an “off” phase of no CAR-T administration for about 14 to about 28 days.

5. The method of claim 3, wherein the dose of the CAR-T cells administered to the subject is about 60×10⁶, 80×10⁶, 100×10⁶, 120×10⁶, 140×10⁶, 160×10⁶, 180×10⁶, 200×10⁶, 300×10⁶, 400×10⁶, 500×10⁶, 600×10⁶, 700×10⁶, 800×10⁶, 900×10⁶, 1000×10⁶ or more cells.

6. The method of claim 3, wherein the dose of the CAR-T cells administered to the subject is from about 0.35×10⁸ to about 14×10⁸ cells.

7. The method of claim 3, wherein the dose of the CAR-T cells administered to the subject is from about 1.4×10⁸ to about 7×10⁸ cells.

8. The method of claim 3, wherein the dose of the CAR-T cells administered to the subject is about 1.4×10⁸ cells.

9. The method of claim 3, wherein a dose of the CAR-T switch administered to the subject is from about 0.01 mg to about 0.1 mg per kg of body weight.

10. The method of claim 9, wherein a dose of the CAR-T switch administered to the subject is about 0.01 mg, 0.025 mg, 0.03 mg, 0.04 mg, 0.045 mg, 0.05 mg, 0.055 mg, 0.06 mg, 0.065 mg, 0.070 mg, 0.075 mg, 0.085 mg, or 0.095 mg per kg of body weight.

11. The method of claim 9, wherein a dose of the CAR-T switch administered to the subject is from about 0.045 mg to about 0.075 mg per kg of body weight.

12. The method of claim 9, wherein a dose of the CAR-T switch administered to the subject is about 0.06 mg per kg of body weight.

13. The method of claim 1, wherein the CD19-positive malignancy is a CD19-positive B cell cancer.

14. The method of claim 1, wherein the CD19-positive malignancy is a relapsed/refractory B cell malignancy.

15. A method of treating, arresting growth of, and/or promoting regression of, a CD19-positive relapsed/refractory B cell malignancy in a human subject, comprising (a) administering to the subject one dose of about 0.35×10⁸ to about 7×10⁸ CAR-T cells, wherein the CAR-T cells comprise a CAR sequence set forth in SEQ ID NO:6, and then (b) administering to the subject a CAR-T switch molecule during one or more cycles that each comprises (i) an “on” phase of about 5 to about 9 days and (ii) an “off” phase of about 14 to about 28 days, wherein a daily dose of about 0.045 mg to about 0.075 mg per kg of body weight is administered during the “on” phase, and wherein the CAR-T switch molecule comprises an anti-CD19 Fab antibody that comprises a light chain sequence and a heavy chain sequence set forth as SEQ ID NOs:15 and 16, respectively; thereby treating, arresting growth of, and/or promoting regression of, the CD19-positive relapsed/refractory B cell malignancy in the subject.

16. The method of claim 15, wherein the CAR-T cells are autologous to the subject.

17. The method of claim 15, wherein the infused dose of the CAR-T cells is about 1.4×10⁸ cells, and the daily dose of the CAR-T switch infused during the “on” phase is about 0.06 mg per kg of body weight.

18. The method of claim 15, wherein the “on” phase is about 7 days, and the “off” phase is about 21 days.

19. The method of claim 15, wherein the number of cycles of CAR-T switch infusion is 2, 3, 4, 5, 6, 7 or more.

20. The method of claim 15, wherein the subject receives lymphodepletion preconditioning prior to being administered the CAR-T cells and the switch molecule.

21. The method of claim 20, wherein the lymphodepletion preconditioning is chemotherapy with cyclophosphamide and fludarabine.

22. The method of claim 15, wherein the CD19-positive relapsed/refractory B cell malignancy is selected from the group consisting of diffuse large B-cell lymphoma (DLBCL), follicular lymphoma (FL), chronic lymphocytic leukemia (CLL), small lymphocytic lymphoma (SLL), mantle cell lymphoma (MCL), marginal zone lymphoma (MZL), hairy cell leukemia (HCL), primary intraocular lymphoma, Burkitt lymphoma, and Waldenström macroglobulinemia.


SEQUENCE LISTING (16 sequences)

SEQ ID NO: 1 - protein, 14 aa, synthetic construct
NYHLENEVARLKKL

SEQ ID NO: 2 - protein, 126 aa, synthetic construct
NYHLENEVARLKKLGGGGSDIQMTQSPSSLSASVGDRVTITCRASQDISKYLNWYQQKPGKAVKLLIYHTSRLHSGVPSRFSGSGSGTDYTLTISSLQPEDFATYFCQQGATLPYTFGQGTKLEIK

SEQ ID NO: 3 - protein, 120 aa, synthetic construct
QVQLQESGPGLVKPSETLSVTCTVSGVSLPDYGVSWIRQPPGKGLEWLGVIWGSETTYYNSALKSRLTISKDNSKNQVSLKMSSLTAADTAVYYCAKHYYYGGSYAMDYWGQGTLVTVSS

SEQ ID NO: 4 - protein, 110 aa, synthetic construct
QAVVTQEPSLTVSPGGTVTLTCGSSTGAVTTSNYASWVQEKPDHLFRGLIGGTNNRAPGVPARFSGSLLGGKAALTISGAQPEDEAIYFCVLWYSDHWVFGGGTKLTVDG

SEQ ID NO: 5 - protein, 113 aa, synthetic construct
QVQLQQSGPGLVKPSETLSITCTVSGFLLTDYGVNWVRQPPGKGLEWLGVIWGDGITDYNPSLKSRLTVSKDTSKNQVSLKMSSLTDADTARYYCVTGLFDYWGQGTTLTVSS

SEQ ID NO: 6 - protein, 478 aa, synthetic construct
QAVVTQEPSLTVSPGGTVTLTCGSSTGAVTTSNYASWVQEKPDHLFRGLIGGTNNRAPGVPARFSGSLLGGKAALTISGAQPEDEAIYFCVLWYSDHWVFGGGTKLTVDGGGGGSGGGGSGGGGSGGGGSQVQLQQSGPGLVKPSETLSITCTVSGFLLTDYGVNWVRQPPGKGLEWLGVIWGDGITDYNPSLKSRLTVSKDTSKNQVSLKMSSLTDADTARYYCVTGLFDYWGQGTTLTVSSESKYGPPCPPCPDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 243 aa, synthetic construct
QAVVTQEPSLTVSPGGTVTLTCGSSTGAVTTSNYASWVQEKPDHLFRGLIGGTNNRAPGVPARFSGSLLGGKAALTISGAQPEDEAIYFCVLWYSDHWVFGGGTKLTVDGGGGGSGGGGSGGGGSGGGGSQVQLQQSGPGLVKPSETLSITCTVSGFLLTDYGVNWVRQPPGKGLEWLGVIWGDGITDYNPSLKSRLTVSKDTSKNQVSLKMSSLTDADTARYYCVTGLFDYWGQGTTLTVSS

SEQ ID NO: 8 - protein, 13 aa, synthetic construct
ESKYGPPCPPCPD

SEQ ID NO: 9 - protein, 27 aa, synthetic construct
FWVLVVVGGVLACYSLLVTVAFIIFWV

SEQ ID NO: 10 - protein, 41 aa, synthetic construct
RSKRSRLLHSDYMNMTPRRPGPTRKHYQPYAPPRDFAAYRS

SEQ ID NO: 11 - protein, 42 aa, synthetic construct
KRGRKKLLYIFKQPFMRPVQTTQEEDGCSCRFPEEEEGGCEL

SEQ ID NO: 12 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 33 aa, synthetic construct
RMKQLEPKVEELLPKNYHLENEVARLKKLVGER

SEQ ID NO: 14 - protein, 5 aa, synthetic construct
GGGGS

SEQ ID NO: 15 - protein, 233 aa, synthetic construct
NYHLENEVARLKKLGGGGSDIQMTQSPSSLSASVGDRVTITCRASQDISKYLNWYQQKPGKAVKLLIYHTSRLHSGVPSRFSGSGSGTDYTLTISSLQPEDFATYFCQQGATLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 223 aa, synthetic construct
QVQLQESGPGLVKPSETLSVTCTVSGVSLPDYGVSWIRQPPGKGLEWLGVIWGSETTYYNSALKSRLTISKDNSKNQVSLKMSSLTAADTAVYYCAKHYYYGGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC